(19) United States
(12) Patent Application Publication
(10) Pub. No.: US 2010/0174228 A1
(43) Pub. Date: Jul. 8, 2010

(54) Hypoglycemia prediction and control

(76) Inventors: Bruce Buckingham, Palo Alto, CA (US); Günter Niemeyer, Mountain View, CA (US); Darrell M. Wilson, Sunnyvale, CA (US); Fraser Cameron, Palo Alto, CA (US); H. Peter Chase, Denver, CO (US); Eyal Dassau, Goleta, CA (US); Hyunjin Lee, Watervliet, NY (US); B. Wayne Bequette, Albany, NY (US); Francis J. Doyle, III, Santa Barbara, CA (US)

Correspondence Address: LUMEN PATENT FIRM, 350 Cambridge Avenue, Suite 100, PALO ALTO, CA, 94306, US

(21) Appl. No.: 12/589,701

(22) Filed: Oct. 26, 2009

Related U.S. Application Data
(60) Provisional application No. 61/197,230, filed on Oct. 24, 2008.

(51) Int. Cl. A61M 37/00

(52) U.S. Cl. 604/66

(57) ABSTRACT

A system for predicting hypoglycemia based on continuous blood glucose monitor values is provided. The hypoglycemia detection algorithm is a set of individual alarms that are combined through a voting system into one combined alarm. The system could have five components and an overall voting algorithm that produces a binary alarm outcome depending on the number of constituent algorithms that report an alarm. A controller system automatically shuts off the insulin pump when pending or real hypoglycemia has been reached. The algorithms operate in a closed loop and automatically take action when the subject is asleep.

### CROSS-REFERENCE TO RELATED APPLICATIONS

The present application claims priority from provisional application No. 61/197,230 filed on Oct. 24, 2008, which is incorporated herein by reference.

### FIELD OF THE INVENTION

The invention relates to blood glucose monitoring methods and devices. In particular, the invention relates to continuous subcutanous blood glucose monitoring methods and devices utilizing hypoglycemia prediction methods.

### BACKGROUND OF THE INVENTION

Patients with Type 1 diabetes are at risk for severe nocturnal hypoglycemia. Seventy-five percent of diabetic hypoglycemic seizures occur at night. Severe hypoglycemia can be prevented if the pump discontinues insulin infusion, based on the trend analysis of continuous glucose monitoring readings. Real-time continuous glucose monitoring devices with FDA approval are presently available to the general public. One of the major perceived benefits of real-time glucose monitoring is the ability of these devices to have alarms for hypoglycemia. For a real-time alarm to be effective, it must awaken a sleeping subject. However, it was observed that children wearing the device failed to respond to alarms at night. One possible correction of this problem would be to have the sensor send a signal to the pump so that it will stop infusing insulin when pending or real hypoglycemia has been reached and the patient has not responded to alarms. The present invention addresses these needs.

### SUMMARY OF THE INVENTION

Patients with Type-1 diabetes are at risk for severe nocturnal hypoglycemia. Seventy-five percent of diabetic hypoglycemic seizures occur at night. The underlying idea of this invention is that severe hypoglycemia can be prevented as a result of discontinuing a subcutaneous insulin infusion based on trend analysis of continuous subcutanous glucose readings.

The invention provides multiple hypoglycemic prediction algorithms based on continuous subcutaneous glucose values. The algorithms can be used individually or in any combination. In one exemplary embodiment there could be five unique hypoglycemic prediction algorithms: (1) Linear projection, (2) Kalman filtering, (3) Hybrid IIR Filter, (4) Statistical Prediction, and (5) Numerical Logical Algorithm. An overall voting scheme can be used in the case of two or more hypoglycemic prediction algorithms. In one example, the voting scheme could produce a binary alarm outcome and need for stopping insulin infusion based on the number of constituent hypoglycemic prediction algorithms that report an alarm.

### DESCRIPTION OF THE INVENTION

The objective of this invention is to tune the integration of 5 hypoglycemic prediction algorithms based on continuous blood glucose monitor values so that temporarily discontinuing an insulin infusion will significantly decrease hypoglycemia (glucose values <60 mg/dl), with a secondary aim of not increasing the time spent above 180 mg/dl overnight. The Hypoglycemia Detection Algorithm is a set of individual alarms are combined through a voting system into one combined alarm. With each new continuous glucose monitor (CGM) datum, each individual alarm will run independently and will indicate hypoglycemia or euglycemia. Then if the number of individual alarms that have gone off in the last 60 minutes is above a preset voting threshold (V), the voting alarm will trigger. A low voting threshold will generate more alarms, giving more warning but less accuracy. Finally, the combined alarm will trigger if either the voting alarm or the threshold alarm goes off.

Overview of hypoglycemic algorithms:

The hypoglycemic prediction algorithm system includes five component prediction algorithms:
1) Linear Projection (LP): This alarm uses a 15 minute linear extrapolation and uncertainty threshold based on the standard deviation of the glucose measurements in the previous 15 minutes.
2) Kalman Filtering (KF): A Kalman Filter is used to obtain an estimate of glucose and its rate of change, which are then used to make predictions of future glucose levels. The filter is tuned to trade off between the probability that a measured glucose change is real versus the result of signal noise.
3) Adaptive IIR Filter (AIIRF): Infinite Impulse Response Filters update parameters adaptively using the CGM signal. The IIR filter considers a bandwidth of past data to update the filter parameters.
4) Statistical Prediction (SP): (also referred to as the Stanford alarm)—Multiple empirical, statistical models are used to estimate future blood glucose values and their error bounds. From these a probability of hypoglycemia is generated and thresholded to produce an alarm.
5) Numerical Logical Algorithm (NLA): NLA feeds a 3 point calculated rate of change and the current value into logical expressions to detect impending hypoglycemia. NLA provides insensitivity to sensor signal dropouts and easy tuning.

An overall voting algorithm produces a binary alarm outcome depending on the number of constituent algorithms that report an alarm.

Further details of the various algorithms and other details and variations of this invention are described in U.S. Provisional Application 61/197,230 filed on Oct. 24, 2008, which is incorporated herein by reference in its entirety.

1. A hypoglycemia prediction and control system, comprising:
(a) a computer system having multiple prediction algorithms, wherein each of said prediction algorithms on said computer system are configured to independently produce an individual alarm to indicate onset of hypoglycemia based on input data to said computer system, wherein said prediction algorithms comprise in any combination a linear projection algorithm, a Kalman filtering algorithm, an adaptive IIR filter algorithm, a statistical prediction algorithm, or a numerical logical algorithm;
(b) said computer system having a voting scheme for determining on said computer system a single alarm predicting onset of hypoglycemia based on said individual alarms; and
(c) a controller communicatively coupled to said computer system and configured to automatically shut off an insulin pump in response to said single alarm.
